(19) United States
(12) Patent Application Publication
(10) Pub. No.: US 2023/0056585 A1
(43) Pub. Date: Feb. 23, 2023

(54) ANCHORING DEVICE FOR SOFT TISSUE REPAIR

(71) Applicant: TEKNIMED, VIC EN BIGORRE (FR)

(72) Inventor: Stéphane GONCALVES, BALMA (FR)

(21) Appl. No.: 17/799,549

(22) PCT Filed: Feb. 12, 2021
(86) PCT No.: PCT/IB2021/051167

§ 371 Date: Aug. 12, 2022

(30) Foreign Application Priority Data
Feb. 14, 2020 (FR) 2001485

(51) Int. Cl. A61F 2/08

(52) U.S. Cl. CPC A61F 2/0811; A61F 2002/0852; A61F 2002/0888; A61F 2210/0057; A61F 2220/0075; A61F 2002/0817

(57) ABSTRACT

Disclosed is a surgical device for soft tissue repair, including: a flexible anchor consisting of a textile braid forming a first arm and a second arm extending distally away from a vertex area; and at least one suture thread having a median portion placed near the vertex area and two strands passing through the braid transversely at several sites. The first strand passes through the braid alternately at two sites adjacent to the first arm and at two sites adjacent to the second arm, the sites passed through in sequence on the same arm moving away from the vertex zone. The second strand passes through the braid at least at two sites of the second arm, the sites passed through in sequence moving away from the vertex zone. The device is inserted into a hole made in a bone and assumes a compressed configuration after insertion.

### CROSS-REFERENCE TO RELATED APPLICATIONS

This application is the U.S. national phase of International Application No. PCT/162021/051167 filed Feb. 12, 2021, which designated the U.S. and claims priority to FR 2001485 filed Feb. 14, 2020, the entire contents of each of which are hereby incorporated by reference.

### BACKGROUND OF THE INVENTION

### Field of the Invention

The present invention relates to the field of surgical equipment dedicated to the repair of soft tissues and more particularly relates to implantable devices for attaching tissue to a bone structure.

It pertains to a device comprising a textile braid and suture filaments arranged to form a flexible anchoring system the deployed configuration thereof allowing insertion into a hole in a bone, and the compressed configuration of which ensuring anchoring in the hole and the attachment of tissue to the bone.

### Description of the Related Art

A tendon is known to be a structure formed by a bundle of collagen fibres connecting a muscle to a bone. Although they have high strength, tendons are at risk of tearing, either as a result of trauma or due to their degeneration. The first situation is more prevalent among individuals in physical occupations and among sportspersons, while the second rather more concerns older people, often over 50 years of age. In fact, repetitive movements and effort, such as when training high-level sportspersons or in certain trades or manual activities, can cause a variety of injuries such as tendonitis and weakening of the tendon, which can tear or break.

Whenever possible, surgery is recommended in order to reattach the torn tendon to the bone. A first technique relies on treatment with transosseous tunnels, which is aimed at reattaching the torn tendons to the bone by passing filaments through the bone in order to attach the tendons. A second technique consists of repair by suture anchors, during which the tendon is sutured directly onto the bone using a device comprising an anchoring element associated with one or more suture filaments. The anchoring element will hold the device in the bone while the filaments are attached to the end of the tendon to be reattached. This technique is both very simple to carry out with, inter alia, a shorter surgery time, and not very invasive with less damage to the soft tissues, which speeds up the time for recovery and healing. Other types of tissue may undergo this type of repair, in particular ligaments.

Different anchoring devices may be used. Rigid suture anchors are known, produced from metal or reabsorbable or non-reabsorbable polymer materials. They suffer from the disadvantage of occupying a large volume, which makes it necessary to remove bone material, thereby weakening the structure and making it necessary to limit the number of insertion points. This results in reduced attachment. In order to remedy this disadvantage, it is possible to use flexible and deformable anchors. The principle is to insert the anchor with the aid of an inserter into a hole prepared in the bone (or made with the aid of the inserter) and then to pull on the strands of the suture filaments so that the anchor folds or bends, forming a bulky mass which locks itself against the cortical wall of the bone. Then it only remains to attach the suture filaments to the tendon. These anchors are generally produced from synthetic material, which may, for example, be in the form of a textile strip, or alternatively a solid or tubular braided cord.

The manner in which the anchor and filaments are combined is paramount in obtaining a strong and reliable attachment which is easy and quick to install. In particular, it is necessary for the device to have an initially extended configuration with a small diameter, which is capable of being readily installed on an inserter and inserted into the bone. It is also essential that the anchor deforms without difficulty once placed in the hole of the bone under the effect of the traction on the filaments, developing a substantial volume in order to remain firmly locked when it is installed. In fact, the capacity for deformation of the anchor's width is paramount in obtaining good resistance to tearing.

Numerous models for expandable flexible anchors have been proposed. As an example, U.S. Pat. No. 9,826,971 describes a device comprising a flexible strip of textile fibre and at least one suture filament passing through the strip at several points, from end to end, the ends of the filament being free, with or without a knot. The flexible strip folds when it is put into place. Application WO 2019/032797 describes a similar anchoring device, but it additionally comprises an element produced from a spongy material such as a sponge fabric produced from cellulose fibre which increases in volume when it is in contact with an activator such as water. In U.S. Pat. No. 9,974,534, a flexible anchor having a hollow tubular structure is produced from a nonwoven material. A suture filament passes into and out of the tube of the anchor in the proximity of the ends, the anchor being folded in two and implanted in this folded configuration. In contrast, US 2014 277133 proposes using a rectilinear tubular anchoring implant, a suture filament passing backwards and forwards through the wall of the implant from one end to the other. In EP 2 662 030, a braided anchor configured as a V to form two arms joined by a point is associated with a suture filament which passes from one end of the braid to the point and then from the point to the other end, passing through the meshes of the braid at several points. The filament has a collar for tightening the assembly after installation.

However, those devices suffer from various disadvantages in relation to the attachment between the suture filament and the anchor, to the attachment of the anchor to the bone, or from other disadvantages resulting from the configuration of the assembly, which do not offer optimum conditions for the surgeon in charge of the implantation, and finally for the patient. There is therefore a need for an attachment system that withstands being placed under tension as much as necessary when it is implanted in the bone. An assembly constituted by flexible materials is sought which can easily be introduced into the bone and then tightened simply by pulling on the suture filaments.

### SUMMARY OF THE INVENTION

An objective of the present invention is to overcome these disadvantages by proposing a device for repairing soft tissues based on an original assembly of a textile anchor with suture filaments which offers optimum performance as regards the mechanical properties of tensile strength, ease of implantation for the practitioner, and a minimum removal of bone material so as not to weaken the attachment. In accordance with the invention, the device is in an extended initial configuration which is thin and therefore easy to insert, but which can expand in width to a large extent so as to lock the textile device by bearing on the interior wall of the cortical bone of the bone receiving the implant. In an original manner, the expansion of the device is obtained by the effect of two combined actions, namely on the one hand, folding of the anchor into several agglomerated loops, and on the other hand the lateral expansion of the braiding of the anchor. In doing so, the suture filaments can slide without jamming along the entire length of the anchor, which has been shown to be useful for positioning the implant, in particular when making surgical knots. This result is obtained by combining a flexible anchor constituted by a braid of textile material with suture filaments passing through the material at several points.

More specifically, the invention concerns a surgical device for repairing soft tissues, which is capable of being inserted into a hole produced in a bone in an initial deployed configuration and of adopting a final compressed configuration after insertion, the device comprising, in its initial configuration:
a flexible anchor constituted by a braid of textile fibres, which is curved into a hoop to form a first and a second arm extending distally from an apex zone,
at least one suture filament, a median portion of which separating a first and a second long strand each having a free end, the median portion of the filament being placed between the first and the second arm in the vicinity of the apex zone of the anchor, and the two strands of the filament passing transversely through the braid from one side to the other at a plurality of sites,
   the first strand passing through the braid alternately at two sites adjacent to the first arm and at two sites adjacent to the second arm, the sites passed through in sequence on the same arm being increasingly distant from the apex zone,
   the second strand passing through the braid at at least two sites of the second arm, the sites passed through in sequence being increasingly distant from the apex zone.

The device in accordance with the invention is described here and in the remainder of the description in its initial deployed configuration, i.e. spread out or developed in its entire extension, as it is presented to a surgeon wishing to repair a tissue (tendon, ligament or the like) by attachment to a bone. The compressed configuration which it can adopt once put in place will be detailed below.

The anchor is produced in the form of a braid of textile fibres, i.e. fibres which are capable of being braided. The term “braid” should be understood to mean a type of braid in which all the fibres are interlaced with one another in their turn, in a relative diagonal direction, as opposed to a woven braid. Different ways of braiding are possible. As an example, flat braids and round, solid or tubular braids exist, produced from a variable number of fibres. In accordance with the invention, the fibres are flexible and braided with a moderate tension so that the anchor is itself flexible. As a result, it can be bent into a hoop and it offers an easy passage between its fibres so that a suture filament can pass through it. Furthermore and essentially, it can be deformed in order to adopt a compressed configuration and carry out its anchoring function.

In its initial configuration, the anchor is curved into a hoop, forming two arms which extend distally from a tip or apex zone and which visually define an interior space and an exterior space of the hoop. The tip of the hoop can be engaged on an insertion tool to push the device along its axis into the receiving bone. At least one suture filament passes over the anchor braid. In a variation which will be described below, the device may comprise a second suture filament. The suture filaments are associated with the anchor by passing through the braid at different sites, which are generally interstices existing between the fibres of the braid. Thus, there is no need to pierce the anchor at the locations where the suture has to pass through.

Said at least one suture filament is threaded onto the anchor by passing through the braid in the vicinity of the apex zone. The portion of the suture filament between the anchor arms is designated as the median portion, from which two long strands extend. The two strands are of comparable length so as to provide two free ends which are easy for the surgeon to handle. It will, however, be understood that insofar as the suture filament slides on the braid, the median portion is not strictly defined as such, but corresponds to a segment of the filament occupying the location situated between the two arms, at a short distance from the tip of the anchor.

The two strands of the suture pass through the braid transversely from one side to the other at a plurality of sites, each in accordance with a particular pattern. Irrespectively of the method of braiding the retained anchor, the strands of the suture filament pass through the braid in their entirety substantially perpendicular to its main axis, without penetrating the latticework of the mesh.

The first of the two strands passes through the braid at two sites adjacent to each other, passing alternately from the first arm to the second arm, the sites which are passed through successively on the same arm being increasingly distant from the apex zone. The alternating passage from the first to the second arm expressly indicates an alternation, i.e. a repeated succession in space which causes a periodic motif to gradually appear, in a regular order. In the present case, it is a question here of alternating the pairs of sites through which the first strand passes. Each passage in two adjacent sites of an arm corresponds to a passage towards the exterior of the hoop followed by a return towards the interior between the arms of the braid. The pairs of adjacent sites are progressively increasingly distant from the tip of the anchor. They can be totally offset so that each site is further from the tip than the previous site, the suture filament passing through the interior space diagonally, or in fact the separation may be only between two adjacent sites of the same pair, in which case the suture filament passes through the interior space perpendicularly to the axis of the anchor.

The second of the two strands passes through the braid at at least two sites of the second arm, the sites which are passed through being increasingly distant in sequence from the apex zone. This strand therefore always passes through the same arm of the anchor, the first passage being made towards the exterior of the braid, then towards the interior, at a site which is further from the tip of the anchor, and so on.

In accordance with a feature of the device of the present invention, the first strand passes through the braid in 2n+1 pairs of adjacent sites, alternately of the first arm and of the second arm, with n being equal to 1 or 2. In other words, the first strand passes through three pairs of adjacent sites or five pairs of adjacent sites, on one arm and on the other in alternation. Preferably, three pairs of adjacent sites are passed through, and preferably also, the progression of the strand towards the end of the arms of the anchor takes place by the spacing between two adjacent sites, the suture filament passing through the interior space of the hoop perpendicularly to the axis of the anchor. Finally, the path of the first strand through the braid terminates by opening into the interior space of the hoop.

Thus, in accordance with a preferred feature of the device of the present invention, the first strand passes through the braid at two adjacent sites of the first arm, at two adjacent sites of the second arm, and at two adjacent sites of the first arm, in sequence and in this order, two sites which are passed through in sequence on the first and the second arm being at a substantially identical distance from the apex zone.

In accordance with another preferred feature of the device of the present invention, the second strand of the suture filament passes through the braid at two sites of the second arm, one in the vicinity of the apex zone and the other in the vicinity of the distal portion of said second arm. As a result, the second strand terminates its path through the braid by opening into the interior space of the hoop.

In accordance with a preferred feature of the device in accordance with the invention, the sites of the second arm through which the first strand passes are located between the two sites of said second arm through which the second strand passes. By way of illustration, it could be said that the second strand straddles the first strand when it passes to the exterior of the hoop.

In a preferred embodiment of the device in accordance with the invention, the free end of the first strand and the free end of the second strand each open out from a site situated at the distal portion of one of the arms of the anchor into the space comprised between the two arms in a manner such that the distal ends of the first and second arms form fins spontaneously adopting a diverging orientation with respect to the axis of the anchor. These diverging fins tend to move further apart on either side when the suture filament is placed under tension, and therefore contribute to better locking of the anchor against the wall of the receptor bone.

Preferably, in order to promote this locking, the distal ends of the first and second arms forming the fins are stiffened by thermosetting. As an example, before assembling the device, it is possible for the ends of the braid to be exposed to a temperature in the range 150° C. to 400° C., depending on the textile material used.

The device as described above can be introduced into a hole in a receiving bone, leaving the strands of the suture filament free on the exterior. Traction on the strands will cause it to acquire a compressed configuration: the braid retracts on itself and widens thereby and forms a globular cluster of loops, while the fins move apart, the assembly bearing in the hole and against the cortical wall of the receiving bone. The assembly of the braid and of the suture filament therefore constitutes an anchoring device, but it is the textile braid which acts as the anchor per se because of its deformation under the action of the traction exerted on the strands of the suture filament, the free end of the strands being fixed to the tissue to be secured to the bone.

In a particular embodiment of the invention, a second suture filament is threaded onto the anchor, passing through the braid at two sites adjacent to each other, at the level of the apex zone. The filament passes through the two sites from the exterior of the hoop, so that one segment of the filament covers the apex zone of the braid, this segment being designated as the median portion of the second filament, from which two long strands extend. The two strands are of comparable length so as to provide two free ends which are easy for the surgeon to handle. It will, however, be understood that insofar as the suture filament slides on the braid, the median portion is not strictly defined as such, but corresponds to a segment of the filament occupying the tip of the anchor. The two strands then extend between the arms of the anchor.

Thus, according to a variational embodiment of the device in accordance with the invention, the device comprises a second suture filament, a median portion of which separates a first and a second long strand each having a free end, said strands passing through the braid transversely from one side to the other at two sites of the apex zone which are adjacent to each other, in a manner such that said median portion covers the apex zone and such that said first and second strands extend between the arms of the anchor.

In accordance with a preferred feature of the device in accordance with the invention, the strands of the second suture filament are interlaced with the portions of the first suture filament passing transversely between the sites of the first and second arms. By way of example, the strands of the second filament pass successively above then below the transverse portions of the first suture filament, it being possible for the two strands to intertwine simultaneously in phase or, in contrast, separately in an offset manner. The strands of the second suture therefore remain orientated between the arms of the anchor, in a good position for the surgeon to employ the device. When the two strands intertwine simultaneously, the transverse portions of the first suture filament are slightly pushed back and curve around the two strands, in a manner such that a passage appears along the two strands, facilitating the introduction of an insertion tool up to the apex zone of the anchor.

A surgeon installing the implant will pull the two strands of the first suture filament to fold the arms of the braid, but also the two strands of the second suture filament which will bring the tip of the anchor towards the cortical wall and force it to agglomerate together with the folds of the arms in order to generate a more voluminous agglomerate, ensuring reinforced attachment.

Obtaining a bulky cluster is also ensured by the choice of the type of braid constituting the anchor. For this reason, in accordance with a preferred feature of the device of the invention, the anchor is constituted by a braided tubular sheath, taken in a flattened configuration along its longitudinal axis. The flattened sheath therefore forms two superimposed braiding thicknesses of braid. Tubular braids are known in the manufacture of surgical anchors, but they are routinely used to pass one or more suture filaments through the central lumen, unlike in the present device. Now, unexpectedly, it has been found that, by deforming, the fibres of a tubular braid can confer a more pronounced effect of expansion when a filament passes through the entire thickness of the braid. The tubular braid is flattened into a braided double layer which will compress in accordance with two processes. On the one hand, the braid bends, forming folds which are ordered to a greater or lesser extent, and on the other hand, the braid itself flares laterally, giving an additional volume to the compressed anchor. Finally, an excellent capacity for expansion is obtained, which expands the thickness and width of the anchor. This makes it possible to use anchors of smaller size which ensure optimum mechanical strength and therefore secure attachment.

In practice, it is advantageous to assemble the anchor with the suture filament or suture filaments from a braid that has been flattened beforehand, which can easily be carried out by a mild heat treatment, for example of the order of 150° C. It turns out that this arrangement further improves the device, by promoting a certain rigidity of the braid and by allowing the suture filaments to pass through the meshes of the braid and slide. Thus, preferably, the sheath is flattened while hot before cutting to a selected length, of between 2 cm and 5 cm.

The sheath is formed from a plurality of textile fibres. The braiding parameters are adapted as a function of the number of filaments and their count, in order to obtain a moderately compact sheath, allowing the passage of a splicing needle between the meshes while maintaining a high expansion power for the braid. The sheath may be formed, for example, from 4 to 6 textile fibres measuring from 20 dtex to 200 dtex (dtex: mass in g of 10000 m of yarn). The person skilled in the art knows how to modify the braiding parameters, such as the point or the number of patterns per unit of length for this purpose by acting on the settings of the braiding machine.

As regards the suture filaments, they may conveniently be selected from those which have already been approved, for example, by the United States Pharmacopoeia (USP), of the monofilament or braided type, which may or may not be resorbable. Braided filaments have the advantage of having a higher coefficient of friction, which secures the knots. Filaments with a diameter of between 0.4 mm and 0.7 mm, for example, are suitable. It is specified that in all the embodiments of the invention presented above, said at least one suture filament and the second suture filament, if it is present, can slide over their entire length with respect to the braid of the anchor.

Numerous materials may be used to manufacture the device in accordance with the invention, among which are, quite naturally, the synthetic polymers which are suitable for medical devices and more specifically for surgical use. Polyethylenes may be used in particular, whether they be ultra-high molecular weight polyethylenes (UHMWPE), ultra-low molecular weight polyethylenes (ULMWPE), high tenacity polyethylenes (HTPE) or standard polyethylenes (PE). Polyethylene terephthalates (PET) or in fact polylactic acids (PLA), the latter being resorbable, may also be used. The person skilled in the art will be aware of these materials and be able to obtain them from the manufacturers to the required specifications.

Thus, in accordance with one feature of the invention, the device may be produced from a resorbable or non-resorbable polymeric material which is identical to or different from the anchor, for said at least one suture filament, and for the second suture filament, if present. Advantageously, said polymer material may be selected from polyethylenes, polyethylene terephthalates and polylactic acids.

The surgical device described above in its initial extended configuration must be placed on an insertion tool and pushed either directly into a receiving bone, or into a hole which has already been drilled or punched with the aid of a tapping punch, in order to introduce the anchor into the hole in its entirety, leaving the strands of the suture filament or suture filaments free on the exterior. This configuration has a first length (excluding free strands) and a first width which is sufficiently small to enable the device to be inserted into a hole in a bone during surgery on a human or animal, with minimal removal of bone material. Once the inserter has been removed, the surgeon will pull on the strands to cause the braid to deform. The latter retracts on itself, thereby widening and forming a globular cluster of loops while the fins move apart. The device finally acquires a compressed configuration: the arms of the anchor are folded and the braid is compacted along its axis. This configuration has a second length which is less than the first length, and a second width which is greater than the first width. The assembly provides an anchorage which bears in the hole and against the interior cortical wall of the receiving bone.

Thus, in accordance with a feature of the surgical device for the repair of soft tissues which forms the subject matter of the present invention, it has:
an initial deployed configuration, in which the arms of the anchor are extended and the braid is extended along its axis, the configuration having a first length and a first width allowing the device to be inserted into a hole in a human or animal bone, and
a final compressed configuration, obtained by pulling the strands of said at least one suture filament, in which the arms of the anchor are folded and the braid is compacted along its axis, the configuration having a second length which is less than the first length and a second width which is greater than the first width, providing the device with its anchoring configuration in the hole.

The assembly of the device in accordance with the invention ensures a good distribution of the filaments and a balanced bulk. The lateral expansion is substantial, the diameter of the final configuration being of the order of twice the initial configuration (increasing, for example, from approximately 5 mm to approximately 10 mm). The security of the attachment is ensured both by the presence of the fins and by the double thickness of the braid, which prevents any possible tearing. Sliding of the strands is retained, even after tightening. The mechanical properties of tensile strength have been tested successfully. It should also be noted that the assembly of the device does not give rise to any particular difficulties; this is essential for industrial manufacture.

A device in accordance with the invention is particularly useful for repairing soft tissues to be fixed to a supporting bone of the upper limbs (shoulder, elbow, wrist, hand) as well as the lower limbs (hip, knee, ankle, foot). This repair may consist in reattaching the tissue after sectioning or tearing, in reinforcing it after an injury, or in reconstructing it. In the shoulder, interventions may, for example, concern repair of the rotator cuff, injuries to the labrum, the capsules, instability of the anterior shoulder, Bankart lesions, lesions of deltoids, acromioclavicular repair, capsulolabral reconstruction, etc. The device may also be used for tenodesis of the biceps, for replacement of the tendon of the biceps at the elbow, for reconstruction of the tendon of the biceps, of the ulnar and radial collateral ligament, for the repair of lateral epicondylitis, etc. The device may also be employed in the repair of the middle of the foot, forefoot and ankle, to the reconstruction of the Achilles tendon, of the hallux valgus, as well as to the repair of lateral or medial stabilization, etc.

### BRIEF DESCRIPTION OF THE DRAWINGS

The present invention will be better understood and its details will become more apparent from the description below of different embodiments, made with reference to the accompanying figures, in which:

FIG. 1 shows a device in accordance with the invention, in which an anchor and a suture filament are assembled in accordance with a first embodiment.

FIG. 2 shows a device in accordance with the invention, in which an anchor and a suture filament are assembled in accordance with a second embodiment.

FIG. 3 shows a device in accordance with the invention, in which an anchor and a suture filament are assembled in accordance with a third embodiment.

FIG. 4 shows a device in accordance with the invention, in which an anchor and a suture filament are assembled in accordance with a fourth embodiment.

FIG. 5 shows a device in accordance with the invention in which an anchor and two suture filaments are assembled.

FIG. 6 shows the same device after positioning on an insertion tool for its implantation in a receiving bone.

### DESCRIPTION OF THE PREFERRED EMBODIMENTS

### Example 1

FIGS. 1 to 4 show a surgical device in a first variational embodiment in the deployed configuration (i.e. extended configuration, before installation), which comprises an anchor 1 assembled with a suture filament 20. The anchor 1 is constituted by a braid 2 curved into a hoop. The hoop comprises an apex zone 3, or tip, from which a first arm 11 and a second arm 12 extend in the distal direction, which define an interior space and an exterior space of the hoop which can be identified visually. The braid 2 is a tubular sheath, produced with the aid of synthetic fibres, produced here from very high molecular weight polyethylene. The tubular braid 2, initially with a round cross-section, is flattened by hot pressing, for example at a temperature of 150° C. for 15 s to 30 s, in a manner such that it comprises a double thickness of interlaced fibres, forming two layers which are perfectly superimposed and integral. A braid with six fibres with a count of 50 dtex, with approximately eight motifs per cm, makes it possible to obtain a suitable rigidity of the anchor 1 and a spacing of the meshes allowing the passage of a splicing needle for assembly with the suture filament 20. The ends of the braid 2 may be thermally fixed, for example by means of cutting to the desired length using a heated knife.

The suture filament 20 is associated with this anchor by passing the braid 2 transversely from one side to the other at a plurality of sites 50. Here, a suture filament of the standard type, produced from UHMWPE polyethylene with a diameter of USP 2 (0.500 mm-0.599 mm), was used. It was threaded through the meshes of the braid 2 which move slightly apart when a tool such as a splicing needle passes through it, without it being necessary to pierce any perforations. The suture filament 20 is first stitched into the braid at two sites 50, one on the first arm 11, the other on the second arm 12, in the proximity of the apex zone 3, for example 5 mm from the extreme tip of the hoop. It is passed from the interior towards the exterior of the hoop in a manner such that a portion of the suture filament 20 is placed between the first and second arms in the vicinity of the apex zone 3 of the anchor. This portion, known as the median portion 23, separates a first strand 21 and a second strand 22 of the suture filament 20. Each strand passes through the braid 2 transversely from one side to the other at a plurality of sites 50, then terminates in a free end 211 and 221, with a length of several tens of cm (for example from 20 cm to 70 cm), allowing ligation of the soft tissues in a convenient manner. These lengths and positions are given for a device in the state in which the surgeon receives it, but they may be modified during its handling, in particular by sliding or gliding the suture filament 20 relative to the braid 2.

The arrangement of the suture filament 20 relative to the anchor 1 satisfies several criteria, with common points and possible variations relating to each of the two strands 21, 22. They have in common that they each pass through the braid 2 an even number of times, progressing from the vicinity of the apex zone towards the distal portion of the arms 11, 12. Since they begin their passage over the braid by passing through it towards the exterior of the hoop, they always end it by opening towards the interior of the hoop. The free strands 211, 221 of the suture filament 20 are therefore situated between the distal ends of the two arms 11, 12. These distal ends spontaneously adopt a diverging orientation relative to the axis of the anchor 1, forming two fins 112, 122 about 5 mm long, for example. These fins tend to move away from the axis of the anchor, even more so as the strands 21, 22 are placed under tension. They therefore fulfil a function of attachment to the cortical wall of the receiving bone of the implant, allowing better stabilization in situ and increasing the tear strength. They may undergo a treatment with a view to hardening them further (by hot pressing, impregnation with a polymer, coating with adhesive, etc.).

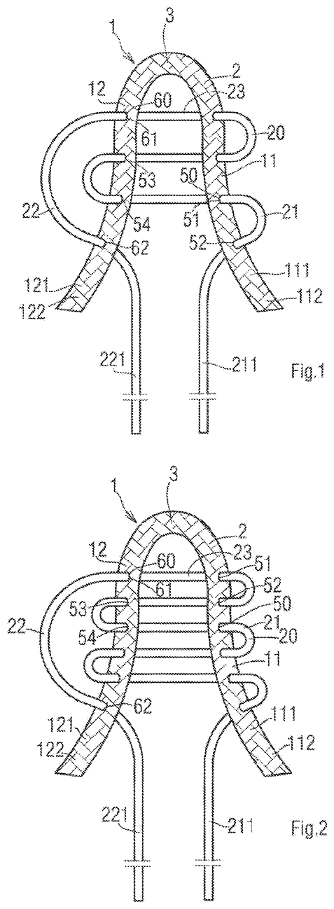

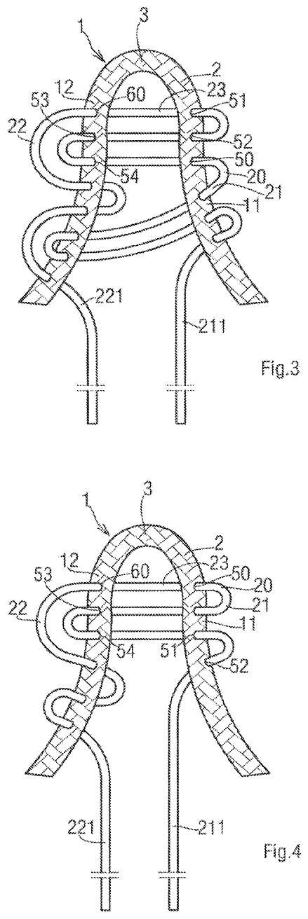

Each strand 21, 22 advances on the braid along a different path. The first strand 21 penetrates the braid 2 at two sites 50 adjacent to each other, located on one arm, then penetrates the braid 2 at two sites 50 adjacent to each other, located on the other arm, several times. More precisely, it initially passes through two adjacent sites 51, 52 of the first arm 11, then through two adjacent sites 53, 54 of the second arm 12, then back to two adjacent sites 51, 52 of the first arm 11, and so on, always towards the exterior and then towards the interior of the hoop and progressing from the vicinity of the apex zone towards the end of the arms 11, 12, up to the distal portion 111 of the first arm 11. In accordance with an alternative embodiment shown in FIG. 1, the first strand 21 passes through the braid 2 in three pairs of adjacent sites, alternately from the first arm 11 (two pairs of sites 51, 52) and from the second arm 12 (one pair of sites 53, 54). The same applies in the variation shown in FIG. 4. In accordance with alternative embodiments shown in FIGS. 2 and 3, the first strand 21 passes through the braid 2 in five pairs of adjacent sites, alternately from the first arm 11 (three pairs of sites 51, 52) and from the second arm 12 (two pairs of sites 53, 54). Preferably, the sites 50 of the same arm are substantially equidistant. The distance separating two adjacent sites 51, 52 or 53, 54 corresponds to the progressive advance of the strand from the apex zone 3 towards the distal portion of each arm. In contrast, as illustrated in FIGS. 1, 2 and 4, two sites 50 passed through in sequence on the first arm 11 and the second arm 12 (and conversely on the second arm 12 and the first arm 11) are at a substantially identical distance from the apex zone 3. As shown in FIG. 3, this distance may be greater. Whatever the case, the path of the first strand 21 ends at the distal portion of the first arm 11, towards the interior of the hoop.

The second strand 22 of the suture filament 20 for its part penetrates the braid 2 several times at two sites 60 situated on the second arm 12. More precisely, it passes through two sites 60 of the second arm 12, two or four times, always towards the exterior and then towards the interior of the hoop and progressing from the vicinity of the apex zone towards the distal portion of the second arm 12. In accordance with an alternative embodiment shown in FIG. 1, the second strand 22 passes through the braid 2 at two sites of the second arm 12, namely a site 61 in the vicinity of the apex zone 3 and the other site 62 in the vicinity of the distal portion 121 of said second arm. The same applies to the variation shown in FIG. 2. In these cases, it is preferable for the two sites 61, 62 to surround the sites 53, 54 of the second arm 12 through which the first strand 21 passes, i.e. for the sites 53, 54 through which the first strand 21 passes to be located between the two sites 61, 62 through which the second strand 22 passes. In accordance with alternative embodiments shown in FIGS. 3 and 4, the second strand 22 passes through the braid 2 at four sites 60, which may or may not be interposed between the pairs of sites 53, 54 through which the first strand 21 passes.

### Example 2

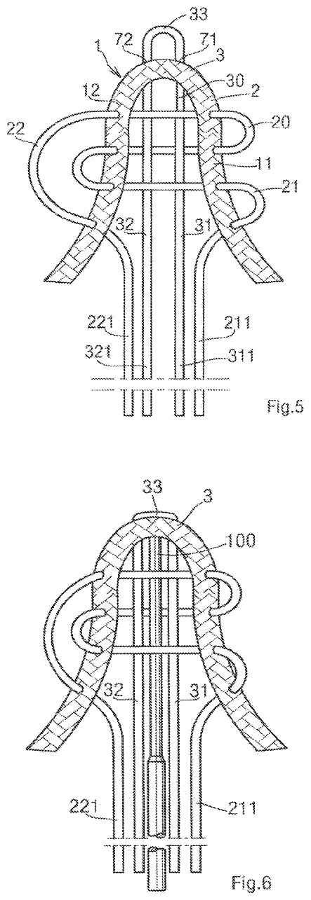

The surgical device in a second variation embodiment may comprise an anchor 1 assembled with two suture filaments 20, 30. The anchor 1 consists of a braid 2 curved into a hoop, and the first suture filament 20 is associated with this anchor by passing the braid 2 transversely from one side to the other at a plurality of sites 50, as described in Example 1 above. FIG. 5 illustrates a device in a deployed configuration, in which a second suture 30 is associated with the assembly shown in FIG. 1. This device may also be associated with the other assemblies presented in Example 1.

The second suture 30 is associated with the anchor 1 by passing the braid 2 transversely from one side to the other at two mutually adjacent sites 71, 72 situated on the apex zone 3 of the anchor 1. A standard type suture filament produced from UHMWPE polyethylene, with a diameter of USP 2, with a colour which makes it easy to distinguish it from the first suture, has been used here. It is threaded through the meshes of the braid 2 which move slightly apart when a tool such as a splicing needle passes through it, without it being necessary to make any perforations. The second filament 30 passes from the exterior towards the interior of the hoop in a manner such that a portion of the suture filament 30 is placed above the apex zone 3, and against the apex zone when the second suture filament is extended. This portion, known as the median portion 33, separates a first strand 31 and a second strand 32 of the suture 30. Each strand passes through the braid 2 transversely from one side to the other at the sites 71, 72, which extends between the arms 11, 12 of the anchor 1, and then terminates in a free end 311 and 321, with a length of several tens of cms (for example from 20 cm to 70 cm), allowing the soft tissues to be ligatured in a convenient manner. These lengths and positions are given for a device in the state in which the surgeon receives it, but they may be modified during its handling, in particular by sliding or gliding the suture filament 30 relative to the braid 2. As illustrated in FIG. 5, the strands 31, 32 of the second suture filament 30 may be interlaced with the portions of the first suture filament 20 circulating transversely between the sites 50 of the first and second arms 11, 12, passing alternately above and below. The routing of the strands 31, 32 of the second suture filament 30 is advantageously simultaneous, so as to create a passage and to provide guidance for an insert 100 as far as the apex zone 3, as illustrated in FIG. 6.

Tests carried out on the anchoring device in accordance with the invention have exhibited excellent results as regards the mechanical properties of tensile strength during their installation. Their properties from the point of view of their expansion in volume and expansion have been tested successfully with regard to the risk of tearing. In addition, the function of sliding before and after implantation, essential for the production of surgical knots, is ensured. Their size is satisfactory, no excess material having been observed. Finally, mounting on the inserter does not give rise to any difficulties.

1. A surgical device for the repair of soft tissues which is capable of being inserted into a hole produced in a bone in an initial deployed configuration and of adopting a final compressed configuration after insertion, wherein in the initial configuration, the surgical device comprises:
a flexible anchor constituted by a braid of textile fibers, which is curved into a hoop to form a first and a second arm extending distally from an apex zone, and
at least one suture filament, a median portion of which separating a first and a second long strand each having a free end, the median portion of the suture filament being placed between the first and the second arm in the vicinity of the apex zone of the anchor, and the two strands of the suture filament passing transversely through the braid from one side to the other at a plurality of sites,
the first strand passing through the braid alternately at two adjacent sites of the first arm and at two adjacent sites of the second arm, the sites passed through in sequence on the same arm being increasingly distant from the apex zone, and
the second strand passing through the braid at at least two sites of the second arm, the sites passed through in sequence being increasingly distant from the apex zone.

2. The device as claimed in claim 1, wherein the first strand passes through the braid in 2n+1 pairs of adjacent sites, alternately of the first arm and of the second arm, with n being equal to 1 or 2.

3. The device as claimed in claim 1, wherein the first strand passes through the braid at two adjacent sites of the first arm, at two adjacent sites of the second arm, and at two adjacent sites of the first arm, in sequence and in this order, two sites which are passed through in sequence on said first arm and said second arm being at a substantially identical distance from the apex zone.

4. The device as claimed in claim 1, wherein the second strand of the suture filament passes through the braid at two sites of the second arm, one in the vicinity of the apex zone and the other in the vicinity of the distal portion of said second arm.

5. The device as claimed in claim 4, wherein the sites of the second arm through which the first strand passes are located between the two sites of said second arm through which the second strand passes.

6. The device as claimed in claim 1, wherein the free end of the first strand and the free end of the second strand each open out from a site located at the distal portion of one of the arms of the anchor in the space comprised between the two arms in a manner such that the distal ends of the first and second arms form fins spontaneously adopting a diverging orientation with respect to the axis of the anchor.

7. The device according to claim 6, wherein the distal ends of the first and second arms forming the fins are stiffened by thermosetting.

8. The device as claimed in claim 1, further comprising a second suture filament, a median portion of which separates a first and a second long strand, each having a free end, said strands passing through the braid transversely from one side to the other at two sites of the apex zone which are adjacent to each other, in a manner such that said median portion covers said apex zone and such that said first and second strands extend between the arms of the anchor.

9. The device as claimed in claim 8, wherein the strands of the second suture filament are interlaced with the portions of the first suture filament passing transversely between the sites of the first and of the second arm.

10. The device as claimed in claim 1, wherein the anchor is constituted by a braided tubular sheath, taken in a flattened configuration along its longitudinal axis.

11. The device as claimed in claim 10, wherein the sheath is flattened while hot before cutting to a selected length comprised between 2 cm and 5 cm.

12. The device as claimed in claim 10, wherein the sheath is formed from 4 to 6 textile fibers with a count of between 20 dtex and 200 dtex which are braided together.

13. The device as claimed in claim 1, wherein the device comprises resorbable or non-resorbable polymer material which is identical to or different from the anchor, for said at least one suture filament.

14. The device as claimed in claim 13, wherein said polymeric material is selected from polyethylenes, polyethylene terephthalates and polylactic acids.

15. A surgical device for the repair of soft tissues as claimed in claim 1, wherein the device is configured to be capable of:
an initial deployed configuration in which the arms of the anchor are extended and the braid is extended along its axis, the configuration having a first length and a first width allowing the device to be inserted into a hole in a human or animal bone, and
a final compressed configuration obtained by pulling the strands of said at least one suture filament, and the strands of the second suture filament if it is present, in which the arms of the anchor are folded and the braid is compacted along its axis, the configuration having a second length which is less than the first length and a second width which is greater than the first width, providing the device with its anchoring configuration in the hole.

16. The device as claimed in claim 8, wherein the device comprises resorbable or non-resorbable polymer material which is identical to or different from the anchor, for said at least one suture filament and for the second suture filament.

17. The device as claimed in claim 2, wherein the first strand passes through the braid at two adjacent sites of the first arm, at two adjacent sites of the second arm, and at two adjacent sites of the first arm, in sequence and in this order, two sites which are passed through in sequence on said first arm and said second arm being at a substantially identical distance from the apex zone.

18. The device as claimed in claim 2, wherein the second strand of the suture filament passes through the braid at two sites of the second arm, one in the vicinity of the apex zone and the other in the vicinity of the distal portion of said second arm.

19. The device as claimed in claim 3, wherein the second strand of the suture filament passes through the braid at two sites of the second arm, one in the vicinity of the apex zone and the other in the vicinity of the distal portion of said second arm.

20. The device as claimed in claim 2, wherein the free end of the first strand and the free end of the second strand each open out from a site located at the distal portion of one of the arms of the anchor in the space comprised between the two arms in a manner such that the distal ends of the first and second arms form fins spontaneously adopting a diverging orientation with respect to the axis of the anchor.
